SEQUENCE LISTING (4 sequences)

SEQ ID NO: 1 - DNA, 20000 bp, Agrobacterium tumefaciens
cttcaccgtctcaggcgtaccgatggtcacggctgtctcttacaaatcctttcaggaactgccgaaagccaaagcaccagctgtccttcaaaagctcgcccaggccgtcgcggcagaaggtttttcaggtatccagatcaacaaggcactgtcgtcaatcgatgcccatcaggaaaccagcggaagtggcaggattcagacgctgcgggttgtcgcccgccagaaaggcgccgctgtccggatcgatgctgtcttcaatattcaggcaggacagatcgccgacaaagacgtcatccgcaagggcatctgcgacatcataaaaggcgcgtaaggcatggcttaaagacactccggtccagcagcacgagccgtcgtgcacaggaagccaatctgtttcgcgcaggatgttacgaaatcgatgattgaagcttatcgctgaccgtatccgcgggtagtctccagtgttagatatgcgccgagcgaactcatcatgccgctacgccaaagacggtcattgatctcacgtgtttggaccatattttctgtgcaaactaaacgatgacatagggcgatttttagtggcggacaaatacagacttcccgaagagttttttaccactcggtttctcgttagacgcatcgtacccacagacgctgaagctattttcgaagggtggaacaccgatcccgaggtgacgaagtacctgacgtggaaaccccactccgagcttggccagacacagcgggcgattgaagaaaattatagtgcgtggaatgcaggtacatcgtttccagctgtcatctgccatcgcgaacggccacatgaactaatcggccgtattgatgcacgtccgatgggccacaaggtctcttacgggtggcttgtccgaagaacctggtggggccggggtgttgcaagcgaggtcgttcaactcgctgtagaacacgcgttatcgcatccgcgcatctttcgcaccgaagcatcctgcgacgttctgaacacggcgtcagcaagagtgatggaaaaagtagggatgacaaaggaggccgtgcttcgacggtacctttttcaccccaatttttcgaatatgccgcgagacgccttcctgtattccaaggtacgttaactcagtgaaatcacggggcgtccaacttcctcataatcccgctattccaagaccggaaaagtacacgtcatccaaaaaagcaatatgtgtctatcgaacgcagggaattaccggattttccagaaagcgacgtcggaaagttcctgcaacaaccacacccgatccaaatcctgaaatcaatcttgctagcaaaaggtcaggtaatgaatatgacaccagccaataatgggtcccaaatgccaagagcgacgtcgcgataaggtaaagttggccgtccgtgcctgggcgacgaaaagccacgaaatgaaagccggcgacgctccccactaggcatgatacaaactgaccgagaattatcgttggttttgcagtgagcccatccagcagacaaaagacgaacgccaagaatccagcaccgagcaactgcaaaacggcggctccaataaaacgtgttccgcagggatatttgggtttcgaatagcgaaaatatgtgaccaagataccctcctctgaagtggtgcctttgaaatcagggcgaccatctcaaaattcgtacaacctcaacttgagttgaggtcgaggggggcggcgtcacaaggattaaccgttgtgtggatagcggcacaaccgggtgtaactgtctccgctttacattttctgactgcctaggcgggctggccaataggaaaaatcgaagccatctggcggcactcaaccgcggcgacacagtcgcgctcaggcaggctagaggtggttgtctaacgttcaacctgcgtcagacagctacctggcataactagattgagccaacagccgagattgcgtcagacgacttctctcaatggggttggtcataccgattgaaacgactggaacccaaaaatcagtgccatcaaactaggcgcgttcgacgttgacggaacacatccaggtgattgtcgaggatggagcgcaccgatccataggttcgcgcgccgatttccaatgcccggccacaggccgcattgaccctttcccgctcaaaagctttgacgagccgaataatgcctaggcaagctctgtagccctgttcgggatgcgggcggtcggcgagaatgcggtcgcagagcaaagcgacgtccgatccgattgccgacgcctcgcggctgatacgctcgatcgtccggtcggcaaaccgccgatgggaagatggcatgtgctccggtgtcgtcgtgtgcttgccattgccactcgatcgcctgtgagctgcgatccgttcgcccttgtagaatatctcgatcgtgttggcagtgatccgcgcctcgacctgctctcgggcaaaccggtagggcacggagtaaaagtgcttgtcgatgtcgacatgataatcgaggcctgcccggcgtatcttccattcagcaaagacatatcgctcgaccggaagcggtcgcagcgcgggatagtcgagctcttcaaacaactgtcgccttgtgcgcccaatgcggcgcaagacgcgtttgtcgttcagatcgtggaggagttcgccaatcgctttgttcacgtcagcaaggctgtagaagacgcggtgacgcagccgacccagtagccagcgctcgacgatgcgaaccgccgcctcgactttcgccttgtcgcgcggccgacgcggtcgcgttggaagaaccgcgctgccgtaatgtgtcgccatcccggtgtaggtgcgattgacctgcggatcaaaatggcatgccttgatgacggcaaccttggcattgtcgggaacaagcagagccggtgcgccgccgaatgtctcaagcgcaagcagatggcattcgatccagtcgggaagggtcccgctccagcgggcatgggcaaatgaaaggctcgatgcgccgagcactccaacaaacacatgggcctgccgcgtcttgcccgacaatctgtcgatgacaacagagaccgtgtcgcctgcataatcgacgaagagcttttcgccagccgcatgatcctggcgcatcgtcacaggcagcttcaaagcccaatggcgatacaaatcacagtaacgggaatagcggtaaccctcaggatgaaggccgatatattcgtcccacagaatctgcagcgtcacatgtttgcgcttcagttcgcggtgaacctgcgcccaatctggctcggcgatgcgccgatgacctgtctttgtcccggcagccttgtaaagggcagcttccagcaccgcatcgctgacatcctcagccaatggccacgacagcccggcacgctccagacggcgcaatgtctcacgcaccgtcgaaggagcagcaccgacgcgcaccgcaatcgacttgtggccgagcccttcttcaaatctgtgtcttaaaatctcgcggacacgccgcatcgctactctctccgctggcattgcgttccttcctctcagcacacctgaaagggacgaacttctcaccagcaggaaaccccgacaaacaccccaatcagggggcgacatcatctcggaacaggggagcgactaattatcggaattggggggcggcatcatttcggaatcagggggcgacatgcctcggaatttgcagtcactcctgacgagcggcaggggtggccctgtgaaacgcaggccgcgccggacgacgaaccactctcaagctctaaccatttatgctgcacacgatctgtgcggcattatttcaattaagccaaagatgccaaacagcaatatctccaggaagatccccgctttggccaccatgcacatatgcgaaatagcggccatgatcggatctagcggcgtccggcaatgaaagcgcgcacttgtacggcaatgacaccactgcatattctggcggggaaagccccattgccgtcgcaaggaacggactgagatggcttgccgtggaggcggtgcccctaacataggcaagcattacggcctctctattagtccaagcatagtcgccggtcgtaaaacggctgagtccattccggcaataaagatcgactgtctttgcctgtgctatgtcgatgagcttgcactcaacgatcaaggggaacaaatgctgtcgaccggttagggaaatttgaatatcgggccgcatctcaaaacgggttgcgtcgtaattcttcgtttctgtgccacgcgaaaccgcgcgaaccagcaaactcgccaaagggtcagcccccaccagatggttcaagcgatcaaccatcaaactgttgaggtgcttctcgtcaccgctagaaactgcagcaggataatccgcttttattcccgtgtaagcctgttgaagcagctccagaatgaattcgagatgcgaatggtcgattgccggtagaggcagttcttgtccacgaatcaattcatacaggtgatcttgcaacccggagatcagtccactcatactgcaggactctccctaaaaaactccggtcgtgtccagattatacgttgcgcggcaaggcgggcctgtgtctcgctccaatatcgagattgggcgaggagcatgatagccaggctgtgcttgccctcatctatgacaatttcagtcgcactggtgcgatcagcaaccgcagtaagagcgcgaatatccgcaattgaatccaccgtctgatcttcggcttcagtgttccagcacgcaacaatacgaagcgcctgccacggggatgattgtcgatcaggtatgtaatcgacacggacggtgcatttgaaacgttgagcaaaccttgtgagttcggtttccagcgttcggcaataggttttgatctggtggatcaacggctcgctctgtgccaattcccgatttgaagcgtaagggagattaaaccgcagtgtatcgtcgataacagtcaggtcccgttcgccgagaccgtagagtttcgctacccaagcatcgacatctggccaaacgtcgccgccctctttttccaaccgcgcaaaaagattggttacctcccgcacccgatcctgcgcgagtgactcgaagggcggaatcgccacctcctcaaggatagatttctcaaccacctcacgctcaaagccgaattcgccgctggttatcagcgatatccagagcgcaacgcgactaccaagcacaagtgcaagatagcgcgtcaataacgacgcctcggcggctcccttggaactaaagccataccaactctcattgaatacgatgtcttgatcgcagactgacatctgtatccgctgtagcttggcaggcggggatttatgaacgagcaagatcggcccgttatagatagctctatctcttgcgcgatgaacgcgatcagtgtcgaaaactggaagctgtgaaggatcgataactaacgttgcggacgacacgtcatcaagatgaggaagtccatgcaggtgagtggcgtcgtcgccgatagtgccatcatcatgcttcttgctactatctctgagcttctgatagccatttccattcgcgagacctccatcgcccaactcgcgccaatagcgtcccagcgtcgtgaaattgcctcgacgaaccctgtcaattagcgaaagatcagcctcactgccgcgaaataggatttttagggtctccgggcgttcccgcaaatcctgcggacggaccacatacgcgttcgtcgcatcgacccgcattacgccggcattattgaaacccttttcatagcggggcgtgagcatccgaaatccggacgctgtatgcgcaggccggttgaccgcaaaaaggaggcaaaacggcgccgaaatgctgggccagacctttgtctggcgtagctcagagccgttgatgatcgaggttacatccatcgcttcgaggaggctctgccgcgccagcggcatgccatccccttgctggaatagaaaccgcgcatgcagcgcaaatatgatttgagcgtcaggttttgcccatcgcatcgcccgccagacaaatggcaaatcgagaactgcgttgggcaaaggaggagcggttataccgtctccgagtctgctccgagcgattttatgaacctccgtaagcaggaggttccagtcttccaatccggtcgcgctggcccacggaggattgccaatcacgagatcgtactggccgtcgtgctcctctccgataagcgagccgaggcttcccaactgctttgcttcgggacgatcaacatcggtggtatcgactggtcgatgcaagacaacaccgcgcaaatcgtcgaagtgcagcttgtccacgggcttgggatttggatcgagttcgatcgatagcaagtagaggccgagcgcgcaaaagcgcaaagcggcctcattgatgtcaaatccgcgcacctgcttgtaaaggatttctcggagcgctgctgtgtccggtctgtgcccttctgatctccagcgcgccgcaacgagttcacgaaacgccgcgagcagaaacacgcccgcgcccgcggctgggtctaatatgcgcgcggccgccgaaataccctgagcattcagtccacgaaacgcggctgataccatcaggtttgcgatcggatgcggcgtataaaagccgccctcctgctgttgctgttttggggcgtgcttacgcaggtaatactcataggcttggctcaacactccaactgttatgtgcgagaagtcgagattatcccactgctctttccaacccagggacaattgccccccctctgcgcgccgcagaatgttgccaatgaactgccccccatttcgaccggacagtcggcataagcagaaaggctcaagcacaggcttgaggacaggcttatgtctaacgactatcgacacgttgaattgctgacgggtgatgttcgccgcaggcggtggacaaccgagcaaaagctgacaatcattgagcagagttttgaacccggcgagacggtatcttcgaccgctcgccgtcatggcgtggcgcccaatttgctttatcggtggcgcaggctcttgagcgagggaggtgctgcagccgtggattctgacgagccggttgtcgggaattcggaagtgaagaaactggaggatcgcgtccgggagttggagcgcatgctcggtcgcaagacgatggaggtcgaaatcctccgcgaagccctttccaaagcggactcaaaaaaacggatatcgcggccgatcttgttgccgaaggacggttcgcgatgaaggccgtcgcagacacgctgggcgtctcccgttccaacctcatcgagcggctgaaaggcagatcaaagccgcgtgggccatacaacaaggccgaggatgcagagcttctgcccgccatccgcaggctggtggatcaaaggccaacctatggctatcggcggatcgccgcgctcctcaatcgcgaaaggcgagccgccgatcagcctgtcgtcaacgccaaacgggtccatcgcatcatgggtaaccacgccatgctactggagcacacagccgttcgcaagggccgcctccacgatggcaaggtcatggtcatgcgctccaacctgcgctggtgctcggacggcctggagttcgcctgctggaatggcgaggtcattcgtctcgccttcatcatcgacgccttcgaccgcgagatcatcgcctggacggccgttgccaatgcaggcatttccggctcagacgtgcgcgacatgatgttggaggcggtcgagaaacgcttccatgcaacccgagccccgcatgctatcgagcatctctctgacaatggctcggcttataccgcgcgggacacgaggctgtttgcgcaagcactcaatctcacgccctgcttcacgccggtcgccagcccgcagtcgaacggcatgtcggaagccttcgtcaaaacgttgaagcgggactatattcggatatcagctctaccggacgcccaaacagcgctccggctcatcgacggatggatcgaggactacaacgaaatccatccccattccgcgctcaagatggcttcccctcggcagttcatcagggctaaatcaatctagccgacttgtccggtgaaatggggtgcactccagccaagaacataacatgcttccaacgtgagcttcggaaacgcagcatccgacaacggcagcaggtcaccattgaacgtcttatcgagccatgcgcaggaaaggcgaactagatctggccggtcaaacagttcatgatcttcgagaccagactcatcgacagaactctcactgccaacaatgctactagttgatggcagcaggccacgatcagcaagaaagcgcatgaagagcgccctgcccaccaatgatatcgcgtcctcgccggagaggccgctattgccttttaaagacgatatggcttggtcaagcaatttcagaattacgccggtgatccaagtcctgtcattaatctccgccgccgggcgcagatttgccaagcgtgcaaaggtaagccagctctcgttatcggcgacaccagcgtcaacgcgagccttggaaagcgaaaggcgatcgagagcgatccgatagacatccaatcttccgggatagaccactccgaggtacggcgcgtcgcctcgcattgcgagaatccgtcgaacccgcttgagccgatcaagatctccgtttagccggtcgccatcgacaaggaatacgagcggagcttcctgccactcgtagacaccttcaaccagtcgcaaatcctcatcatggtcccgagccacgaggatagtcgagtaggcaagcaactctggtgccgaatgatcgaaaagacgaacacccccgcgatcagcccccaattcatccagggcattgtacatgccgatcataagcctgtccccaaccaggggagccagcccggcacagtagcaccaaattccaaaagggagctgaacgccgtcatcgatcgaggtccaatggcacttggcggtcgccgccaaactggccgggtttcgcccgcccgcgaggcttcttcacggtgagttctaccttcgatatcacgccccggaaatcaaccccttgggtgcccatttcagcgaccaacgcggcaatgatcgccaaatggtcgggaacccctcctgaacgggcataatttgaaacagagttagggttcattccaatcaattccgcaaatgccctgatgctcaggccagcgcgcgcgagatcgcggagaaacccctcatagcccatcgctccaaagccctaatcacataattctatgtatactctcacatagaatcatgtgttgcaatccacaaatgcggattgcgatgagaggtggctttttggcggatgcgcttgtgtgaccgagcttctgaaggggcctttagcccacgctaacgcctaacgccgcatttcccttttcgccctcaattatgcgcttaacgccagatgctggaacaggctcttggcgggaggcttcttgccgatcctgacatggcgagccgtgtcggttgccgatgatctgacgccacggcaggatcaggacaagatttccgaacacgcctatcatatcctgtggaagatacagcaaggccacgttccgaagtttctgtggttggattgctgacgacgggcacaagcgttcaagcaaggttgaacgcggccggggcggagccctaccttggccgtagcggtggatcgttttcgcgcgacggcccgtcgcccgagccctcaatcgttcccgtcgtccggccaaagcggggttgggagcttttattgctgccttccaggttgatccgtttccagccgacgacgccagaatttgcccgaacgttcgtaaaccgcttcgggcgtcaacgcattgaagatcttcaaaacgcctttgccgacacacagatttgaagatgcgtcgaaatacgccaaggcttcgtcattgagttggaacaggtggcttgcagcgctttcctgatataaaaagcccactgcttcatactctgccaacatccaacgggctgcttcttcttcggtcatgctttcttcggcgcgacgtagggttgcaacgtatcctcttcaaatacggaagtttcgttggaagcacccttgaaccatttgcagcgataacttcccttgaaattgccgtagtccttttggacttccgaaatcgtcattgctggcccaccggacttaagttgtacaacgtcgccagtcgaaaatttagcttccattatgaggcccctactttgatgttactttcgcctcaatctatgggaaagttcggctctgttccagcttcaaacaagacggattcgatagaccacgtcaaggatcaacgcgtttcttcgtcaaatccgcttccgcacgcgaagccgcctcgcagccgctcgacgcccgcataagctcatttcggccacctgatttctggtcgtaggtcgttagtatattttcgaaaatcggaatttcacaataaatcaatattcgtggctgaccggttgggattgggaaccatctcattcccgttcaccatgaggattgggcacagtaacatcctccggtatttcttgacgaaggctcggccccttggcgagacgtcgccctagcgcagtcacgaaggcgtcgtagcgttcggcagccttggcgcgcgcggcctgagcagctggttccggcagggttggcgtggttgcaagccagaagcgaacaaagacggcgatcatctcgatcgagatgccgacgtcgcgttcaagccgcatcatacgacgatcgacctgatcgagacgcttggtgatggcagcttcctgtcgttcggcggcgtcgggcgagagcgaggattcgatggcggcctccgccaccagcgaacgggaatggtcgcggcgcgcggcatactcggcgagctggcgcatgacggaaggatcgagatagaccgagaggcggtgcttgcggggcggttttgtcatgggtgcctcacagatccatgccgtcgccgggatcgagcgaaacctgccgcgcgacgctctgcatgatcgtgcgcaggcgactggctgtccaatcagaccggggtcaccgaaaccctgatgggcctgacggacaagatggaactttatccgcgccttgctgaatgcatcgaacagatagagccgaccgcctggaacgttgtgctcaaggcaggcatcaagttccatgtcttcgcgataggcgttgccttgttctggtatgcacaagcacaggtgcgctggttcatgcacgaccttggcataggcgcggcaaaagcgctttccatcttcgccggagcatttctgtttgccacagtcgccgcattcgtcgttgcgctattgatcactctcgacttcaaaagcgtcgcgcccgcagcgagctgatccgccgcgccgcctgcggcgtaaccccgagcgttttcgtgaccgccccggcatgaacaagcggcgttgccatgacaagatcaacgagctccggcaacttcgatgaaagatatgtattccgcgtggcgcttccggcctgtgagggatgcggcatttaactgccgccacatcatatatccctcaggcacgccgcatcgtcaacgcaaacgtgcaacccgctttgcacctgttgattttagaattagtgttttatgtcggggatttagcctgtttgattgtagcgttttggcgtactggatcgaagttttggctattgttggatagtgtagcgtaggatttggttgcgggggcctgagtccacggagacttgtaattttcagacggataggatgccgctaaaatgacttctgctttgcgcgaataacaagggcagttggcctctattcctgctgtcgaactcgtttcgatgcgatacctatccattcataaaggatgccctggtccagtttcgaaaagcgcgctcgatgttttaaccactccagcgagtcaccaggattacaacgggcatagtcctcccgaataagtgcctcacaatcttcgcgaagcctggggtcagttctcagtggcaatcaacagtctggtggctgcacgaggcaaagcagtcaatcgccgtgtcgtcccttctgtttcaagcacggaccttagacagttctgccatgatcagcagcgaattgctatccacggcagtgctcacgccaatggcagcagatcttggcgtcacgaaaggcgcggctggtcagtcggtgaaccggctgtcgagcgctgggcttttcgtctaacgtcctcatccatcctacctcgccggccgtcaaatcatttcactgttggggacggttggtacgtacttgacggtgacaaacacgccgcagcgtcggtttcgagcaatatcttcaatgcgtggctgccaccgcgccgactttactgacccaggttcaaagatagcggaggaaacacatcaactcactggtgccaagggttcaatccactgcttgggcgattggcgcaaagcattcgcatgcttggcgggcgacatgccgaaaaccctgccatattctctcgaaaactgggacggactcgcatagccgacctggaaacctgcggttgctgcgtccagaccttcactaagcatcagacgacgagcctcctgaagcctaagctgtgtgcggaattctagtggactcatattggtgaccgctttgaaatgcgcgtgaaacgtagagcggctcatcccggccacttcggcgccagcttccaccgaacaggtctcgcgaaagtgcttcctgatccacacaatagaccgtgcgatctgattgacatgcgcgccagcttgcacgatctgtcgcaacgccgctcctccaacaccggtaatgagacgatataatatctcacgcttcgccagcggtgaaagggcgtcgatatctcgaggcgtgtccagcagcgcaaggagacgcgaagcggcatcaatgataccctcatccaccttgttaagtaacaggccgatagggccgttgattggcgcgtgctgttctgaaggatagcgaattgccagatccgccaactccacggtgtcaaggtcgagttgcatcgaaagataaggtttgtctttcgatgcttcgattaccgatcccatgaccggaagatcaacggaggcgaccagatagctggagggatcatagacaaagcttgtctgcccgagaaccgcctgctttcgcccctgtgcgacgaggcaaagtgtcggctcgtagacgacaggcatcggcaatgtcgtctcggaacagcgaatgagcttcaaccccggcagctgcgtttcaaacgtaccgtcgtgaggtgcatggcgagcaatggtggcttcgatggtgtgttcacttttcataatgcttttatgccatgccttcttaccaacggaaagtctgagcaagcgatatttggacgatcaagcaagcatatcggacattaaaggagtcagtggtctcccacccagaaatcttccatttcccgatagaagcaccgatctcgtcaccgttttccccctggatcgaccagaacacgaaagttgcagcttcagacaattcggcaaggataacggaatatctgtctaccgctaaatcgcattcccggcacatctttctttctgccagctcgaacgcaaagaccgtttgtccacggacagggggcacggccgattaacgatgagtacctgcaagatcgtcaaatagaaaggaaacgccaatgcagccgacagatttgcaagtgagccagcaggtttcccggctcatgccactgtcaagtggttcccaaaccgcaaaccgaaccgaacttccggaaacaattcgggaattctttctgctactaaacaaccgtcaaattgacgcgcttccacagcttctcgcccccggcgctctgatcgttgacggtaaaggtcgaggagacaccaacgcgtctccccgcgagaggctgaacgacacgctttcctttttgcgttcgccggttgtcgtgcttagatctgagccattgttcgaaggttgccgcgttacggtcgtatcaccgtccggcagcgaccttcgatattacaagctgaacttccagatgatctccggaaagattgtgcttttgaaaatcctgccagcaaaaccagacgcttatgactttccgacatctgcccaagtggccatcgtcaatgtttcgtcgcagtgaaccgctaatgggaaaaagtgtccaatcgcgagtgacctgacttccggctgtgtcccggtacacgctcgcttgctccgccgtgggcaaccgcgatcacatgaaacctgacgcgagaggacgacttggcgatggagttcactgtgacctctttgcgtacttccagtcccggcgcggaagagggccgcttttctcttccgcgtggaccacattaattgcctgctaagacggctgaaatgctctccagagcgcgtatgaatcctcaataaagttgtaggcgctcactttgccatctctcaccgagcagattgccgcgaagtcgctaacaaaatctttgcctgttctacgaatgcgatgcttgaaattgccgatgatcacgaccttgtcgccgctttcaaaggtacgctccacttgaaaatcgagcggctcgaaattgacaggaaatagcgccatccaatcgcgcacctgatcccgtccgtgcctgaggccgatggtgggcacatcactggccccttgaaccttccatacaacatcctcagtcaggcattgaagggtcttttcgacatccttcttgaagaacgtctcgagataatgctggacgacctgtgtgggtgtcatctcggtataagttgtcatctgatatctccttgcaggattagttgcgcattaaggggtgattagcgatggccaccggcgaggatgaaaaattcgccagtcgtccagctcgattcgtccgatgcgaggaaaatcgctgcaggtataatgtcctcaggaacgccgagacgcttgagcggcgtctgcgccatgatcatggggccgaagtcaaagtggagaaagcctccggcctgcacgccgtcagtgtcgaccacgccgggcttgattgcgttgacacgaatcttgcggggggcgagctcgtttgacaacgcgcgcgtcaaaccgtcgattgcacctttgcttgccgtatagaccgatgcgttttctgggccgaaaatcgttaccgtcgaggacatgttcacaatgctgccgccctccgccatgtatttcacagcttctttgatcgcgagcaggtagccaaagacattaagctccatatgcttgcggaacagatcgatagacaggttttccagcggctggaagtcatagacaccggcgttgttgaccaggatgtcaacgcggccgaaagctgcgtttgttgccgcaaacagctcttcaacttccgcgggctcactcatgtcggccttcacggcgatggctgtgccaccagcgtcgatgatctcctcaacaactgcttcggccgcagatttgcttgacgagtagtttactacgactgatgcaccctctgcggcgaaacttcgcgcaattgccgcgccaagtcctttggaagcgccggtgatgacagcgattttaccttgcaatcgattgctcatgtttctttccttgaggctcgttggcccattggtttcatgcttggcgtcatgcgtaattttcgccagcgcttatgcaaaagaggcgtaaccgcgtatgtctgtcttggtgcaagagacgcctattcggcgtctctcgtttggatcgacagaatcaagaggtctgagaggtcttgcctgaccacagatccacaggccccaaccggtcagcgccctgcgtctcgagcatccaacctggatactcgggcggcagctcgcttacagcatccagccgcgcaacttcgtcgtcactcagcacggtgccgaccgcggcgatattatcttcgagctggctaagccgttttgctccgacaatgaccgatgtcacgaccggcttggcgaggagccaggccagcgctattcgtgccgggctgcaaccgtgcgcctttgctatgggcttcaggacgtcgatgacgttccaggcccgctccttgtcgacaatcgggaaatcaaaagaggagcggcgcgagccttccggagactgatttgcgcggctgaatctcccagatagcagcccgccagcaagcgggctccagacaagcaagcccatcttttctgcatcgagcaatggcttgagttcccgttcaaggtctcgacccgcaatcgagtaatatgcctggagtgtattgaatcgttccagcccgaggcgggcagaaacgccaagagccgtcgcaatgcgccacgcctgccagttggatacgccgacgtaacgtaccttgccctgtcgaaccagatcatcaagcgcacgcagagtttcctcgacaggagtgagggtgtcggttgcgtggatttggtacaaatcaatgtgatcggtctgcaggcgcttcaaactggcttcaacagcgtccatgatatgtccgcgcgatgcgccgacgtcgtttcggcctgaacccatccgactatagaccttcgttgccaacacgatctcgtttcgggcaataccgaggttcttgaacgattgtccgagcgttcgctcgctcgcaccggctgaatagacgtcggcggtgtcgaagaagttgatcccggcgtcgatgctggctttgaccaattcgtccgccccggtctggccgacatcgccgatatgctcgtagatgcccgtgccgtcgctgaacgtcatggttccgaggcaaagttgtgagacaagaagtccggtgtttccaagtgtcttgtatttcatgtcagtcgctcctgaagacgcaagcttgcgtacgagccgaagatctcggttcgctgtcgcttgctgatggtagattgatggtccggggcctcgctaagtggtgagacgaatgctcatggagagccccagcaaacgcctcaggtgccaggctgaatctattggcccggcgccccgaagatttcgttgatgtgcagcgagtcggtaaattcccgaccgctcgcgatcttgccgtcgcggaagtgaagaagaaaatggtagttgtttcgatagtgccggccgtccttcatcggcagatcacccttcgcgacgaccgcgagcctgtcttcttccccggtgacctcgtggtactccatcttcagagggcccgcggcgtcatccagaagtcgaggtaagttctcgaggtaagggcgcttcgcatgcgtgccggaaaacttgttgccgggcatgatccaccacgtcatatcgtccgtgctgaggttcgacatggtttcagtgtcgcagcgccccagtgcgtccagaaatgctttggcgattctctctttctgctctctactcatggcgtgtctccttcttagccatttgttgtttgatgttggtaaaatgaggtcgtttcctcgagaagaaaacgcaaaagaatggcggaccgaattccaattttggaaaaccttattatgtgcgggttcagggccgatgtgatgccatcgggcaggtttagccggtcctcgtagatctgcgagacaaacgcgcaagcgtttccagccggtacccaatatcgatcagcaacacgttggagagggcttgctcactggcacaccagttcaatcaacggctcggtaagctacggaaggtggccccgcatcttcgaaaccattgctccaagcgcttagcgcacgtctctaccgagcatgcacctgatgcaaattcggcgagtataccgggaagcttgcgattttcgtcgggacttacatcactgacctggtcgcgacgaaccgccagcccacgacggaagcttcgcgatgaggttcagcttgcaacgcgtattccggtgtgtcgattattgagcacaggtccgcgaagctgtcgtagcggacgatgacgatgtcgtcccatacctcgctctcttttggaagcaatgtcatcaattgggagccagcgtagataagctccgttgaaatattgagcgccgtcgcgatctgctggaaggccgaggcataaccctcatagtatgcggatcgagacgttgtcttcacatgttcgaacccgtcggaatatagcggacggtctctgaagcggaggaagttgatcatgacaaccggcccaccgcctaaccgcttagcagcgtcggcaagtgtttcaggaatgagagcttcaagttccattttttgactttctgacactttatgttgatatgtcagacattgacacttttcgtctttttgtcaagttaggggaatggagaagttttcaaataaagaagaacgggtcctgaccgctgcggaagatgtgttcgcccggtacggattcgcgcgcacgacgatgggcgacatcgcaaaagcggctgccatttcccgtccggcgctctatttaatatttccagacaaagaggcaatcttcacccgtgtgatcgagatgatggacgccagatccctggacctgatccagaatgaggtcgatcagatcgcggctattgatcagaagctgctacacgcctgtactatttggggtctgcatggagtggaactcgctaccgcatatcccgacgctgccgatctattcgatttccaatttccggccgttcgtcaagtttacgaacggtttcagcagtttctcgttcgaatattggaaaaaggaacggcttcttgggaattgccggtgtcacgtgcagactttgcgacgacgctcacctatggtctacgaggccttcgatacgcagcgactgacgttaatcacatgaggcacctcattgaagttcacgtaaccgtgtacggactgacccttacgcgaacggacgcttcaatcggctgagaaaacgatgacgtcaaatccacgtatgctgctcaaccatacgtgcgtgccttcgtttaaatggacttcaagattgcacaactgcggtaccggttttcattgcgttcaggcaatgacaataggattctaagtcactccctcctttttgaaatgctcgatgagtgcgcgaaacgctggcgaagttctgcgtcggctcggataataaaggctgaaacctgggaggtccgttagccaatctgtgagaacctcccggagattgccggcttcaagatcggaggccacttgcgagcgaagcagaaagccaataccgagcccgtccattacggctgatcggatcgccgctccgtcgtcgaggatcaacgctgggtctcccttgtattcaaacttctgactaccctttgccagcggccaccggaatagccgtgacgagctcagatagcgatataatatgcatcggtgaccagccagatgttccggagccaagggaattgggtagttctcaaagtaagatgcagagccagcaatcacgataggggtagacgccgtcaagggaagcgccaccatatctttatcgatgagcgtaccgaaacggatcccaccgtcaaacccttctgtgacgatgtcatccagtctctcactcgtagagatttcgaacacgacatcggggtagcgctgacaaaagcttggcagcattggccgaacgatgctttcaaaggcgaccgggatcatcgtcagccgaacgacgcctgcgatcgtctcccttgcttcctgtaaacgcaggagctcttcccggagatcgcgcagtgccggaccgagtatctccaataggcgtaaccctgagccggttggagccacgcttctcgtggttcttgcaagcagagcaaccccgattttccgttcgagttggctaatcgtgtagctcaaagtggactgcttcacaccaagttcggatgccgcatcggtgaagctctggtgtcgcgcaaccacttcgaagactgctaaatcgcggaggatatgccggtccatttatcgatcctatctatagagcttcttgaggcgcagtatcttatcaatatgcggatggacgtctatgcttcttcgacatcaagttttgtcgacagcctgcaagggatggatcgtgcctctaaacaggtgagttgtgaacgagggcgcgatttgccgccgcacaggcccagcgataattaaaaaggaa

SEQ ID NO: 2 - DNA, 12588 bp, Agrobacterium tumefaciens
gcgcgcccaggaagtagtttgacttccacaatgagcttgcggagcagcgcatggatccgattggtcatgttgccgccctccggcaattcgtcgggagtaaccaactcggacaggggcagggatggaaactcgaggggattagacatatcaaatccataactgagatatcagaagagtttctgacatatcggaatgcgaaatttctgtcaagcggtcagtttgccgcgatatcggcccaaaggacgaagtgcagtgcttagctggttcgaggagcgaaatggctggaaccaagttaaattcgtgagagtgcgcgcaagggcacgatctaaaccaaccctggctttaccggagacagtttgatttaagttatgtgctggtgcgtccagcatggcgtgatatcgttcttcggccacggccggagggatgtttccgatggctccagaaggggcttcggacgtggcggtttgactccatggacgatgacgaatcggagaagagaatgagaaacgcagagcgagcaggtccgcttcggtatcttgaggtatcggccatcggcagtttttgctctgcatttttttatttcggggtggccatggcaatgccggcggacatgacggcggagcgccttctcaatatctgtgaagcgcccaccatgcaggccgcgatgatcaagggtgacgaacttggctggccgcggctgaccgccgcggaaacggaggaatggcgtcgtagtttcgtcgcatataatgagggttcggtggtggtcgtgggctggcggggcgagaacgccggcagagccgagtcattgtctttttgggttgcgactggtccaaacggacacaaggcatgcgcctattccacggcaaggcctgccggttttctggatgccttgtcggagcggcttggtgcaccggataatctcgacaaaaatgacgcgatagaaagtacgacagcctggtggaaacgaggtgcggtcgagtattcttttgtccagatcggctcatccgctgtcgtcaatatccgttcaagtcagtgaggtagtggcgtaaacttcaggtaaattgtcgcataatccgacaggccgcttcccgtggaacttaagattgtcagccaagctggcgctcaattgcgaccttgtcgaggaccgaccagaccctacggatttttgcctgttcgaagtcgtagaaaacatgctcgcagaattgaacgcgccggccattgaccggaaggtccatgaagatggatttgggcgtacagtcaaaaaacagccgggcggcaagccgcgtcgcgtcgctgacaagaatctcggcctcaaatcgcaggtcgggaatgtcggcaaagtccttgaccagcatatcgcgatagcccgacagcccgaacggccggccattgtgttcgacattgtcgtcgacgaaggtgccgagttcatcgaaggcttggtgattaagggagtcgaggtaagcgagatagatgtcgttgagtgtttgcaagttgtcttcctctaccagtatggtttgtgctgcccgcaactctggagttaccgaatcatactggtgcaggtcaaactgtgatgagctcgcacctgccatcatctagccggtaaaacccatcctgcggaacgggtagctcgagcatttcctgctttaaaagcccgagaaaaacaccccgaaggatccgcccgaacagaccatgggaaatggcgatggtcggatgccggacatccaggatccatgacgtcgcgcgcttgcaggaatcatcaaaactttcgccatctggtgccctgaaataccagtcgaacgcattggacccgtcaagatggccgggaaactcgttgtcgatttcaaaacgcgtcaacccgtcccaggaccctgtcgtgacctcaaccagtcggtcatcttcgatatgcgggagcggcagcttcgcctgtactgtacccgttctgccgtttgtcgcacgcgcccgagcggactgatctgcatctggaacaactggttatcattgttgagtgcgtcgcggagcaggcgagcaacctggtccgcctgttcaacgccccgtggcgtcaagggtgaatccaattgcccctgaaaccggccaagagagttccagagagtttcgccgtggcgaaggaggtaaatggtcggtagagccacgcttgtttcatcctgcacgttcgttatcgattgctgtcactaggaagcatcaatacggaaacgtcccgattgcagcgcattgatggccgactgcaaagcctgatccgtgtccttgcctgacactggcaggacatggtgctcaaaggcgccgagatcggcgaactgggagtgaagatcggcgaccaccagtggatcgctgaggctgtcgccaccccggtccagacagcgttcgatcgcctcagccgctgtcgtgcgcagcacaatgtaatgcagcggccgggcaagtgccgtaaaggccggcagccagtccggtcggacgacgccgtcaaggatgacgaagtagccctccttggcgtaacgaccggcaacatcggcggcgatctgcatgatcatgcggttctgctgatgggattgcggcagccatggatcgatgcggccgtgcttgatatatccccacagatcatcgctgtgaaaatgcacctttggaacgccgggaaggttcgctagcgcttcggcgattgtggatttgccagagccggggtgcccggagagaagcaggatattaccgccaagatcgtccgtcatgttcattcactgacgttgggcatgcgtaaagcggctaatgatgtaatcattcgtctatcgacgttaccaccctggtgtttcctatcttggctcgcaggcaaatgaggccccctatctggcaaagcttatcacaagataagcagacgccagaagcagggtgacgattgtaaagaatatcagacctgccgccagttcatcgaactggccgtcgcgtccattgcggtcccagtcgtaagccatagccaacctccatcgttgctgtaagggaagggtggtcccgtatggttaacgaaagcctaatggcggcaggctgaatgtttccggtatgatctgatatctgggtcgtagcgaccgtttcgtaacgagacgagttgcaaaggaggcacagaagtaataccgttggttacgacggtattacggaaatttgacatgacaacaacggtaacggccaaagggcaggtcaccattccaaaagctgtacgcgagcttttagggatctcaccgggaagctcggtcgattttgtccgggctcccgatggacggatcgttctcgtcagggcggacaagaaacagccactgacgcgttttgctaagctgcgcggacatgccggtgaaggtcttggtaccgacgccatcatggctttgacccgtggtgacgagtgacgcttgtcgatacaaatgtgctgcttgatcttgtgacggacgacccggtttgggccgattggtcaatcgagcagctcgaactggcaagcgtttcaggttcgctgtacatcaatgacgttgtctacgcggaactatctgttcgatatgagcggatagaagagcttgacgcttttgttgatcaggcggggttgaagttcaccccttttcctcgcgcagcgttatttctggcaggtaaggcctttaccaagtatcaccggggcggcgggacccgtaccggtgttttgcctgacttttttatcggcgcccacgcagcaatacaaaaccttcccttgttgacgcgagatgtggctcgttaccgatcgtattttccaactgtcactttgatctcacctgaagtttaggaatgcgcgggccttagcacttgtgagccgacttcggttttcgaagtagtttcgcgctcttcgcctgaggtcaggcgagccgcaatgctttcaactggctggcttgacgaggctgatttcggttttgctgtcgaggacgatatgcaggtcgcggatcatgccggctgcctgcagcagggcgtttcgcacgtcgtcgtcgctgcgccatgacgtcgcgaattatctgtcacaagatgaatattttgacgaggcagagaccgcgagcttctatatcaggcacatgccaaagcaggcgttgcgttcgaccgatccgattcacgactacttcactgtccttcttccgagcgtcggataccgcggtggtaacgggcggctgatgtatcaggcgcggctatcggaattatcacgcaatccaaccggcatggttcgcctctttgttgatgcttgccgcgatcttcgggttttttatgcagtgtccggcatgtcgcttttcttctgttcctatgcggcaggtgcgactgaaaaagcatatcctttgtttcgtcggtttccaggccttctttacgaagatggcagcaatttcacgttggaaatattgcgtcgcaccgatgtgatccgagacgtgaattggttgacagccatcaacgatgaactgctggcgcgggtcggtggcttagagaaagcaagagacgtgctgggagacgagattgtcctccatccctacgaaggaggcgtcgttttccaggcgggtgcccgcggcgtaccgcgcagtcaacgattttctcaagcccctgcgttaggaggcctgggattatccctatatatgagcgccttacggtgtcgatgagatggagtgtacgcaatggtggactcatcgatttgacgatggtgaacgttaagctcgcccagcctcaacgatttcatcaccccgatggccatccttaaggagcggaggtgaaacgtctgggcgacactaaggcatctcatacccggagatcattcctgccgataaaacttagttgatttgctaagttttcccttgctccgccacgatgaagcatgatagttagcaatatgactaagcatcatcgcgaactttcactgatcttccaggctctggccgatccgacgcggcgggcgattctggcgcgcctcggcggcgggccggcaccggtcatggagctgtccgctcccacggggctgcgtctgcccacggtcatgcggcacctttccgtgctggaggaggcggggttgatcatcacgtccaaggatggtcgggtgcgcacctgcgccatcgtgccggaggcgctggagccggtatgcacgtggctcgatgagcagcgggcgatgtgggagagccggcttgaccggctggaggcatttgcaatgcaggccatgaaggaggattccgaatgacaacgaaaccgggtcaacaaagcgcacacgctgaaccgcatcagccacaagacggttttgcgacgctcagctttgaacgggaaattgccgttccgctatcggctctctggcaggtctggctgtcacccgccgcccgggcggtgtgggcttctccctcaccctcggtcaccgtggagttcctggaggcggacagcaggctgggcggtcgcgaagtgtcgctctgcaaggtcgccggccagccggatattcgctgtgaatgcggctggctggagctgcagccaacccgccgcagcgtgaattacgaggtggtctcatccggcggcgtaacccagtcggcagcgctggtcactgccgattttcagtctgcggaagagcggagccgcttgaccgtaacggtgcagctttcctctctggccagggatatgcgcgatggctatcacgaaggtttcggcgcgggtctcaacaatctggccagcgtggccgggcggaccatggtgctggaacgagtgataaaggtgccgcgaaacatcgtctggaaagcctggatgaacgagaagacgctgccgcaatggtggggccctgaaggcttttcctgccgcacgaagaggattgatctgcgcaccggcggcgaatgggtctttgacatgatcggccctgacggcacagtcttcccgaaccatcatcgttatgtcgagatccggcctgaagagcggcttgcctatacgctgctgtggggcgaaaacggtccgaaacatgccgatgcctgggcctccttcgaagatcaggatggcgcgacgaaagttgtgctgggcatggtgttcagcacggacgccgagttccagcaagcgaagggtttcggcgccgtggagctgggtcagcaaacgctgggcaaactggagcgcttcgccaagtttttttaaatggaccgatcctgcatcgacattttctatatccgaaatccgagctcctttatactctgtttcttgatcgttattcggcgataaccttgatttataggatactcaatgaatttataggaggccaacccaaatatggtggtgagcgctaaagcacatagagccattatccctagtgcgactaagccgggcgtaaaaaatagacctaccgctttttcagaaacttgaatcattaaattgtgaacgagatagattgaataacttgcgtttccaaggtggataacgagtctatttcgcgcaaccaagccgttttcctcaaggaatacgcacccggagataagcattaaaccagctaatccccaagtcattggtcgaaatatattgttactataacttagacccaaaccgttgcccgtgacagcaataataagtgctccactggcaattaaaatccaagcaattgttggattggctgaaggagaattttcgcgtctcgaaacataaaaagcaccaaatgctgctcctatggcaaaatcaaggattactgggctggtataatattctagatatgggtttttcgcgagaattattcctgaactagccaggcaaatcagaattgttaccaaaagcgctattctcattccgaaattaggaacaaaaagtagcaaagcaaacagcgaatagaagaaaatttcaaaattcagcgtccaaccaacagacaaaattggctcccacaggccgttgcgcgtgaatgggataaagaaaagagatttccaaatgtagctgagttgcagatcgatcacgccaatggggcgaaacccaatgaggaacagggctacgatagccaacgtcattatccagtatataggaacgatacgaataattcggtttaagaagaattttgccgggtctctgtctttcccgaacgtggtcaacaccataatgaatccactgagaacgaaaaatatatcaaccccggcggctccgaattcatagactttggcccccggaaacaaacgatctacgtagggtaagaaatgatgaaagaatactaggaaagccgccactgctctcaaagcctgcagattgataatcattttatggcctcaaaataccgtcgtagaaaattatcgaatagtaacctatcggtctaaaatattgctctaatttgatattatagtgggacgaagttgttctattactctctaaagccctattctcagaaattttttatctaatcaattgtcgtcacgcctacttcaagaactgccttgaaggagatagtctgactggtgacttttatgaaactgaaatggtggcgcgcaaaaagtaaatggttggacggctgccaagaagcaaaatccttttcctgaagaggtacacaaatttctaatgaatttgcaagctgagaatgatggacccgcggcaacagccctagcgacgcgagggttcttgattagagtggattcctagagtaaaagttttttttgatatttttgatattttttttaaaagattggaatgacaatgagtgcactcgttgtgccgtcatttttctatcgcaagtttctgacgatagctgcattatttaaaaacaacaagcggtggcgccaacccgacgaatggatacgtgtgcacgttttttttcctgtaacttcggtggatggggaaaggctgactcgtgtggtcatgcggcgaggcgaggccaacaactatgaataccgggccttaaacgaggaagaaaaagaagagatgttttgcgatctagcttggtgagacgtcatagcattattgcatgaggcttttcaaattgcatcgacgaaggcttaacaaattgcggcgttacaatgatctacctagcttcattgaagctttaccattatatccgacgcggataattgtatcccgaggtattgtccgtttatatatatttttgtttggtttcttgtcccagtgtaatgtaattccgacagcctttggattgccatttcttagaattacagacatgttagtcatatcgacgctgccaatattgtatttggtatatataaggctatttaattcggcgctgattttgactcatattgctccgattatgtttgcattcttaacctgtctattttttttcaccgataggcggcaaggcgaactatattcgtctgtctttacatttttatatttactttattttcttccctttacgtttgctttgctccgtgaggggtgcttggatttgttttgttggggaatcctggcaggattcgcgacgacgacaatttttttgttgattgatttgtatgttccttacactcttacgaagataggtctcgcccttgtgtttgactatgaagcggtcgctgacgcagcggcaaagggcgactacaatgcgcctctgcttcgcttcgaaaaagccggcggactgtggacgcacggaaatgaagctggtccagtgtttgccctagccgctgccgctgcggcgtctttaacggaaaggcgtcgcaatttttttatatttgcatcatttgtagcgatataccttgtttcgtttagcgccacactaaatagatccgggatgggtacggtcgtattgataggactgatatcgtacatgtcttcgttctcgaaccgaaaaataactttgacattggtttatggctgcattggaacattaattggctcaatggtgattttctttggctcgttcgatctactcgatggcgcgatctcaaagagatttcttgaggacgaaaatgctagctcgaatattttcgaacggcttgaatctctcgggtatggcatacaagtggccctccataacccttttggtataggacttgccgctaggatggctgcaatgaatgcgttcagtaacttggggacgcctcacaatggttttgtttcgactgcttttacttcgggtattttagttggtttttttgttgtgtcctcagtcgcatatatgttgtttcgttatagaaaggtgcgcttttttctttacgtggcagttacactgacttgcggttatttttttgaggaactggacttcaacgccgcctttatgtgttgggccggtttgttgataggttacacctgtttggatctcgattatcgcttggttcgggggcgggcaattttgcggtacttgttgaaactgtttaaaagtcagcagtctgtatgtcctgaagccactagtttatcttccgtgaactcatgaatagaataatagagtgaaaaccatgtgtcgtttccatcaattcaaatattgacgttaggaatctcgtagaggtttaagatgtcacgcgggagcattgtcattttcgtcccaagggtgttggttgatgccttgcgatagaaggtgtctctcgcgtcactactgacttgacgacttttatcgctcaaaaaaggggacattgcgtgtttgatcacttggatttttagttgttctccgggcctgtagtggtttctcattcgttttgtttttcagctacttgacgccaatttgttccttaatttacgcctgctccgctttatatggcgaactttttatttcctcattctttcccatccctcgctggttgacctatattgagccgtgaacccataaacgggcggcgaggcgattaagggcggtcagcctgtaagtaccaaataaaacacatgcggtcgcggacgttgcaaccagcagcgtcgtaagctgcaaaagagtcggcaaacgcttttcgcccttcttcgataaagtcgtaacgaaggctgatatcaaccatctgctgtcgtgcggttgttgcggagagtgcgacgcgctcggcccaagttaaaccgtctggctcgctcatcaacgtgtttacctctacccaataacattcgcaggtagcttccggcagcaggaattgcagtggcccgtcaacgttaagaaggcctaatttctcggcttcattgataaccagagatattgtttcaggttgccaaccccattcaccgccgctgcgacaagcggcagatcgcagtgtatctggaaggagcatctcagggttgtctttcatgcggcaaggctatacatcttccaagagcgatcaacgtctgattcagtgccgtcacgggccgatacgatctgaccgaccacgagtggcgcgtgattcagccgctgttgcccaacaagccgcgaggtgtgcctcgtgttgatgaccgtcgcgcgctgaacggcattttctgggttctgcgatcgggtgctccttggcgtgaggtgccacagcgctatggccccctacatcacctgctacaatcgcttccgacgctggatgaaagccggaatctgggacagcatgatggacggcctcaccagcacatcgcatgatcggatcacgatgatcgacggcacttcaattcgcgtacatcattcagcggcaacattgaggacggatcacccagatcgctgccttggaaaaagtcgcggcggtctcacaaccaaaatccatgctttaaccgatggaaaagggctgccaatcaagattgccattacacccggtcatgcccatgaccttacggcagcgggcgaactactcgataatctttccgtcaggtgcgatgcttcttgcggacaatgcatatgatgccaactggctacgctcaaagatgagcgcgcaacgctcgtgggctaatattccgcgaaagtctaatcaaaaggaggcaatcgtcttcagcccttggctgtacaaaaaagcgcaacctcatgagcgcttcttaacaagctcaaatacttcagacgggttgcaacccgatacgacaaacttggaatgacgtttctcgcaatgacgaagctagcttgcattcgcatcgtactccgtcataacgagtccacggcctagttatggctgctcacaaagaggaatttaagagtacttgcaataatatttggagaattgattttggcaatgttttgataatggataattcatgaaatatattttcttattatcggcttttttgatatttttattttcggcttttatttacttttttccgatctttggcgcggttgtttgtccgccttgttttggttttgtcgaggttgaaggggggatttatattgacgcgagcctaaatgcggatcttgagaaagataagattttgagtaatctcgatgccgcacatttgttattgagagaggtctacggtgaggttgaggctccgctacccgcaatatttttgtgcgtgtccaagaattgcgccacgtacctaggtagacgtggggagaaggcttcgtcgtttgggcattgggctattgttgtctatcgtgatggaaataactctggcattttggctcatgagctttctcatattgagattggctttaggctaggcttttataatatgtcgtctgtccccatttggtttgatgaaggggtagcagtggtcgcatcgcaagatcgtagatatttgaacgttgacccttccggtagactttcgtgtaaagagggtgtcaccgggccggtgatagccgatctcgatgagtggtgtcggcgcgctagcatcggggacgtcggcatatacagcgctgctgcttgcgaagttatgaaatggatggaccgtaggggcaatgaaggttcattggttagactcttggatttattacgatcgggcgagtcgtttgatgtggcttttgaatagtggtgtaggcagttagttttttacgggtttatttcgtttatttcatttcctcgcacatccccaccctttttctctggtttatacttcttccttgttcggcgatgaataggaagacattcctgttttgaaactgaaccagcgccggctgtgagagaaagacgtcgctctcatatttcgcggtctgacaggtaaagtcctctggccgtgaccggaggagagatggaatctggtttgtggtgggcctttctatttctccgaaacgcattggctgaaggacacgctctgacaggcataaaccgctgatgcggggcactgataagtgtctttattttccctaggcggcaattttcagtgccccgttgcaatgcccatcgagctgaaaggctgatggaaaaagagatggagttcacaacacggtcgcagctgcgtcgcgtgataggaaaggtgtgttcaaatgggtctctatgaaatgtacgtccttgctactcgtgcagataccgccctgatgaggagaaggctggcacgatctctttttgccagggcaaagcttcgaaatcggaagctcaagaagtagtatttacgcaaccagaaggagagttttgattcgatggtcaataggaagaggggagggcaggtgagttgcgacctgagcggcttctgcaaacatatatttatataataaccagcgcaaaacatataaaatatatgcgctgttagattgtatatttttgaatgagggcggatgagaatctgcggacaaacctgattttactgacctgcaatttgcggatt

SEQ ID NO: 3 - DNA, 659 bp, Agrobacterium tumefaciens
ttttcaggtatccagatcaacaaggcactgtcgtcaatcgatgcccatcaggaaaccagcggaagtggcaggattcagacgctgcgggttgtcgcccgccagaaaggcgccgctgtccggatcgatgctgtcttcaatattcaggcaggacagatcgccgacaaagacgtcatccgcaagggcatctgcgacatcataaaaggcgcgtaaggcatggcttaaagacactccggtccagcagcacgagccgtcgtgcacaggaagccaatctgtttcgcgcaggatgttacgaaatcgatgattgaagcttatcgctgaccgtatccgcgggtagtctccagtgttagatatgcgccgagcgaactcatcatgccgctacgccaaagacggtcattgatctcacgtgtttggaccatattttctgtgcaaactaaacgatgacatagggcgatttttagtggcggacaaatacagacttcccgaagagttttttaccactcggtttctcgttagacgcatcgtacccacagacgctgaagctattttcgaagggtggaacaccgatcccgaggtgacgaagtacctgacgtggaaaccccactccgagcttggccagacacagcgggcgattgaagaaaattatagtgcgtggaatgcag

SEQ ID NO: 4 - DNA, 787 bp, Agrobacterium tumefaciens
tactacttcttgagcttccgatttcgaagctttgccctggcaaaaagagatcgtgccagccttctcctcatcagggcggtatctgcacgagtagcaaggacgtacatttcatagagacccatttgaacacacctttcctatcacgcgacgcagctgcgaccgtgttgtgaactccatctctttttccatcagcctttcagctcgatgggcattgcaacggggcactgaaaattgccgcctagggaaaataaagacacttatcagtgccccgcatcagcggtttatgcctgtcagagcgtgtccttcagccaatgcgtttcggagaaatagaaaggcccaccacaaaccagattccatctctcctccggtcacggccagaggactttacctgtcagaccgcgaaatatgagagcgacgtctttctctcacagccggcgctggttcagtttcaaaacaggaatgtcttcctattcatcgccgaacaaggaagaagtataaaccagagaaaaagggtggggatgtgcgaggaaatgaaataaacgaaataaacccgtaaaaaactaactgcctacaccactattcaaaagccacatcaaacgactcgcccgatcgtaataaatccaagagtctaaccaatgaaccttcattgcccctacggtccatccatttcataacttcgcaagcagcagcgctgtatatgccgacgtccccgatgctagcgcgccgacaccactcatcgagatcggctatcaccggcccggtgacaccctctttacacgaaagtcta

(19) United States
(12) Patent Application Publication
(10) Pub. No.: US 2003/0096246 A1
(43) Pub. Date: May 22, 2003

(54) Telomeres of agrobacterium linear chromosome

(76) Inventors: Steven C. Slater, Acton, MA (US); Barbara A. Qurollo, Boston, MA (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 09/992,009

(22) Filed: Nov. 26, 2001

Related U.S. Application Data
(63) Continuation-in-part of application No. 09/923,773, filed on Aug. 6, 2001.

(51) Int. Cl. C12Q 1/68; C12N 1/20; C12N 15/82; C12N 15/74

(52) U.S. Cl. 435/6; 435/252.2; 435/320.1; 435/469

(57) ABSTRACT

Isolated telomeres from the linear chromosome of an Agrobacterium tumefaciens are obtainable from a restriction enzyme fragment at the end of said chromosome which is less than 4,000 nucleotide bases and comprises a segment of consecutive nucleotide bases having substantial identity to SEQ ID NO: 1 or SEQ ID NO: 2. The isolated telomeres are obtained by removing more or less of the segment from the larger restriction fragment. Pairs of isolated and distinct telomeres obtained from opposite ends of the linear chromosome are used for linear DNA constructs for use in producing transgenic plants by Agrobacterium tumefaciens transformation. Such constructs act as linear plasmids and comprise at least an origin of replication and terminal regions obtained from telomeres.

### CROSS REFERENCE TO RELATED APPLICATION

This application is a continuation in part of, and claims priority under 35 U.S.C. §120 to, U.S. application Ser. No. 09/923,773 filed Aug. 6, 2001, the disclosure of which are incorporated herein by reference in their entireties.

### FIELD OF THE INVENTION

Included in the disclosure are nucleic acid molecules representing the telomeric region of the linear chromosome of the bacterium Agrobacterium tumefaciens (hereinafter “A. tumefaciens”) and oligonucleotides based on the A. tumefaciens telomeric sequences and constructs comprising A. tumefaciens telomeric regions and methods of transforming plants using such constructs.

### BACKGROUND OF THE INVENTION

A. tumefaciens is a gram negative aerobic rod grouped within Rhizobiaceae in the alpha subgroup of the proteobacteria. Agrobacterium species have a major impact as phytopathogens, and are the causative agent for a number of plant diseases. These diseases affect a wide range of dicotyledonous plants in more than 140 genera worldwide. Transmission of the disease occurs though soil contaminated with the bacterium that enters plants through fresh wounds or natural openings (such as lenticels). Infection results in the transfer and integration of the bacterial T-DNA into the plant genome (reviewed by Tinland, B., 1996, Trends in Plant Science 1:178-184). Within the T-DNA is a set of oncogenes that upon expression result in a loss of cell division control. Treatment is preventative, such as the removal and burning of all infected plants and rotation of infected soil with non-susceptible plants, as once the bacterial DNA is integrated into the plant genome the disease can progress even without the bacterium.

This loss of cell division control is manifested in different ways among Agrobacterium species. A. tumefaciens, for example, causes the formation of tumors at the crown, roots, or branches, known crown gall disease in numerous crop plants such as almond, peach, apricot, tomato, and grape. The proliferation of these tumors interrupts water and nutrient movement up the stem resulting in stunting, discoloration, and plant death. Plants that do survive have heightened sensitivity to winter injury and drought stress. While there is no cure for crown gall disease, preventative measures can be taken. In addition to the aforementioned measures, is the dipping of susceptible plants with the nonpathogenic Agrobacterium radiobacter (a species nearly identical to A. tumefaciens except it lacks the Ti plasmid), which works to prevent the disease by working antagonistically to A. tumefaciens.

Examples of diseases caused by Agrobacterium species are provided below.

| Agrobacterium Species | Host | Disease |
|---|---|---|
| A. tumefaciens | dicotyledonous plants | crown gall |
| A. rhizogenes | dicotyledonous plants | hairy root |
| A. rubi | caneberry | cane gall |
| A. vitis | grape, chrysanthemum | crown gall |

Plant diseases caused by Agrobacterium infection are induced by transfer of a defined segment of DNA, designated T-DNA, from an Agrobacterium plasmid into a plant (Chilton, M. D. et al.,1977, Cell. 11:263-271; Chilton, M. D. et al., 1982, Nature. 295:432-434; reviewed by Ream, W. 1998, In: “Subcellular Biochemistry”, Biswas and Das (eds.) Plenum Press, New York, 365-384; Hansen, G. and M. D. Chilton, 1999, In: Curr. Top. Microbiol. Immunol. 240:21-57). The pTi (tumor-inducing) plasmid is a large, self-transmissible plasmid harbored by infectious Agrobacterium, and Agrobacterium species are pathogenic only when a tumor-inducing plasmid is present. Such tumor inducing plasmids, referred to as pTi in A. tumefaciens and pRi in A. rhizogenes, contain two regions essential for the ability of Agrobacterium to cause disease. These are the virulence (vir regulon) and transferred DNA (T-DNA) regions. The virulence region is comprised of eight operons (virA-H), of which only virA, B, G, and D are necessary for tumorigenesis (reviewed by Hooykaas, P. J. J. and A. G. M. Beijersbergen, 1994, Annual Review of Phytopathology 32:157-179). The remaining vir operons encode genes whose proteins affect the efficiency of tumorigenesis or host range. The T-DNA region is bordered by two direct repeats, e.g., of 23-25 bp, called the left border and right border. These borders delineate the segment of DNA which will be transferred into the host plant. The genes involved in stimulating tumor formation (specifically the plant growth hormones, cytokinin and auxin) as well as genes required for opine synthesis are located between the border sequences. Opines comprise a novel class of amino acid derivatives not normally present in plants, but whose synthesis in Agrobacterium infected plants provides a carbon and nitrogen source for the Agrobacterium. The particular type of opine produced is used as a distinguishing feature for classifying Agrobacterium strains (i.e., octopine, nopaline, succinamopine, agropine, cucumopine, agrocinopine and mannopine).

The molecular processes by which A. tumefaciens infects plants are generally understood (reviewed by Hooykaas, P. J. J. and R. A. Schilperoort, 1992, Plant Mol. Biol. 19:15-38; Winans, S. C. et al., 1994, Res. Microbiol. 145:461-473; Hansen, G. and M. D. Chilton, 1999, In: Curr. Top. Microbiol. Immunol. 240:21-57). The bacterium is attracted chemotactically to a wounded plant by responding to phenolic compounds, such as acetosyringone, released from the damaged plant cells. Acetosyringone triggers the induction of virulence proteins in A. tumefaciens through a two-component signal transduction pathway. This pathway is comprised of a receptor, VirA, and a transcriptional inducer, VirG. Detection of acetosyringone in the environment causes VirA to become autophosphorylated, leading to the phosphorylation of VirG at aspartic acid residue 52 (Jin, S. et al., 1990, J. Bacteriol. 172:4945-4950). Subsequently, phosphorylated VirG activates transcription of the vir regulon by interaction with a DNA consensus sequence, ryTncAaTTGnAaY (the “vir box”), found within the promoter of all vir regulon genes (Winans, S. C. et al., 1987, Nucleic Acids Res. 15:825-837; Pazour, G. J. and A. Das, 1990, Nucleic Acids Res. 18:6909-6913). In addition, the pH of the infection site (Mantis, N. J. et al., 1992, J. Bacteriol. 174:1189-1196) and presence of monosaccharides (Huang, M. L. et al., 1990, J. Bacteriol. 172:1814-1822) also effect the induction of virulence. Monosaccharides are sensed by ChvE protein, and ChvE also functions to activate vir gene transcription through VirA (reviewed by Winans, S. C. et al., 1994, Res. Microbiol. 145:461-473).

After induction of the vir regulon, a single-stranded version of the T-DNA, called the T-strand, is produced via nicking of the lower strand of T-DNA at the Right and Left Borders (Stachel et al., 1986, Nature, 322:706-712; Reviewed by Zupan, J. R. and P. Zambryski, 1995, Plant Physiology 107:1041-1047). This nicking is catalyzed by VirD1 and VirD2 proteins, and VirD2 becomes covalently attached to the 5′ end of the T-strand. The large gap is presumably filled by repair synthesis, allowing production of an additional T-strand (reviewed and discussed by Hansen and Chilton, 1999).

Transfer of the T-strand, with VirD2 still attached, to the plant occurs through a type IV secretion system that is primarily encoded by the virB genes (reviewed by Christie, P. J., 1997, J. Bacteriol., 179:3085-3094). A single-stranded binding protein, VirE2, is also transferred to the plant, although it apparently is transported independently of the T-strand (Binns, A. N. et al., 1995, J. Bacteriol. 177:4890-4899; Citovsky, V. et al., 1992, Science, 256:1802-1804; Gelvin, S. B., 1998, J. Bacteriol. 180:4300-4302). VirE2 coats the single-stranded DNA and, along with VirD2, targets the T-strand to the plant nucleus for integration (Citovsky, V. et al., 1992, Science, 256:1802-1804; Tinland, B., 1992, Proc. Natl. Acad. Sci. USA, 89:7442-7446). The processes required for T-DNA integration into the plant chromosome are not well understood, although both VirD2 and VirE2 probably play a role (Dombek, P. and W. Ream, 996, J. Bacteriol., 179:1165-1173; Rossi, L. et al., 1996, Proc. Natl. Acad. Sci. USA, 93:126-130; Tinland, B. et al., 1995, EMBO J., 14:3585-3595). To date, the plant proteins necessary for these processes are essentially uncharacterized.

The ability of A. tumefaciens to transfer T-DNA to plants has been adopted by biologists as a mechanism for production of transgenic plants. Because this process is unique in biology, and technically and economically important, much work has gone into elucidating the regulatory and functional processes involved in T-DNA transfer. Most of the critical transformation genes are located on the pTi, although a few (such as chvE) are located on a chromosome. A. tumefaciens also contains a second large plasmid, designated the “cryptic” plasmid, that is still largely uncharacterized. An inefficiency in plant transformation by A. tumefaciens is the propensity for constructs to “read through” beyond the T-DNA borders which results in transfer of more than intended DNA.

A. tumefaciens contains two chromosomes, one linear and one circular (Allardet-Servent, A. et al., 1993, J. Bacteriol. 175:7869-7874), and a coordinated physical and genetic map of the A. tumefaciens C58 genome (Goodner, B. et al.,1999, J. Bacteriol. 181:5160-5106) was recently published. The linear chromosome was initially predicted to contain at least one origin of replication, and at least one terminus site. Our sequence analysis has shown that the linear chromosome has a repABC-type replication system, which is characteristic of plasmids. This single origin is located slightly assymmetrically from the center of the chromosome. This arrangement predicts that DNA replication will initiate at a single site and proceed bidirectionally toward the chromosome termini (telomeres), and a specialized mechanism is required for replicating the chromosome termini. There are essentially two mechanisms by which prokaryotes have been previously shown to replicate the telomeres of linear plasmids or chromosomes (Volf, J-N., and J. Altenbuchner. 2000. FEMS Microbiol. Lett. 186:143-150; Casjens et al. 1997. Mol. Microbiol. 26:581; Hiratsu et al. 2000. Mol Gen Genet. 263:1015; Casjens et al. 2000. Mol Microbiol. 35:490; Wang, S-J. et al. 1999. Microbiology. 145:2209-20; Walther, T. C and Kennell J. C. 1999. Mol Cell. 4:229-38; Rybchin, V. N., and Svarchevsky, A. N. Mol Microbiol. 1999 33:895-903; Barreau, C. et al. 1998. Fungal Genet. Biol. 25:22-30).

The first type of telomere is typified by Streptomyces, in which linear DNA molecules have open ends and carry proteins attached to the 5′ end of the DNA molecule. These terminal proteins serve to stabilize the ends and prime synthesis of the second strand, thereby allowing complete replication of the chromosomes (Qin Z. and Cohen, S. N. 1998. Mol. Microbiol. 28:893-903). In the second type of telomere, typified by Borellia and phage N15, the telomeres are covalently closed, and replication proceeds around the end, creating a large, double-stranded molecule with two repeats of the DNA. The two repeats must then be separated. This reaction is best characterized in the N15 system, in which the protelomerase enzyme has been shown to break the double stranded DNA at the telomeres, then re-join the ends of individual molecules to re-create the covalently closed ends (Deneke et al., 2000. Proc. Natl. Acad. Sci. USA, 97:7721-7726). A similar system may be used by other linear molecules with covalently closed ends.

No orthologue of the N15 protelomerase has been identified in the A. tumefaciens C58 genome. However, the DNA near the telomeres are rich in IS elements, including several putative transposases. One or more of these transposases may play a role analogous to the role of N15 protelomerase, in which the telomeres of daughter molecules are separated by a transposase-type enzyme. Depending on the precise mechanism of replication of the telomeres, the transposase may also play a role in allowing replication of the lagging strand near telomeres by joining ends to allow priming of the lagging strand. Such a reaction would be similar to that catalyzed by IS3-type transposases when they form circles (Sekine, Y. et al. 1996. J. Biol. Chem. 271:197-202.) The telomerase would also be involved in separation of the telomeres upon completion of replication.

The DNA has been assembled near the ends of the linear chromosome allowing identification and isolation of covalently-closed telomeres, apparently having hairpin turn ends. The nucleic acid sequences disclosed herein represent the telomeric regions of the linear chromosome which are useful in preparing linear “plasmid” constructs for Agrobacterium transformation.

### SUMMARY OF THE INVENTION

The present invention contemplates and provides nucleic acid molecules comprising a telomeric region of the linear chromosome of A. tumefaciens. More particularly this invention provides isolated telomeres from the linear chromosome of A. tumefaciens wherein the telomeres are isolated from restriction enzyme fragments at each end of the linear chromosome. In particular a fragment comprising the telomere is less than 4,000 nucleotide bases and comprises a segment of consecutive nucleotide bases having at least 90% identity, more preferably at least 95% identity, to SEQ ID NO: 1 or SEQ ID NO: 2. Moreover, each telomere is obtained by removing more or less of an identified segment of consecutive nucleotide bases from the restriction fragment leaving a covalently-closed double-stranded molecule. In a preferred aspect of the invention a telomere is obtained from a terminal fragment comprising consecutive nucleotide bases of SEQ ID NO: 1 which can be cut by any of the following restriction enzymes: ApaLI, AvrII, BamHI, KpnI, NdeI and NotI. In another preferred aspect of the invention a telomere is obtained from a terminal fragment comprising consecutive nucleotide bases of SEQ IS NO: 2 which can be cut by any of the following restriction enzymes: ApaLI, BamHI, EcoRI, MluI, PvuI and SpeI. Telomeres of this invention are preferably provided in a pair of isolated and distinct telomeres obtained from opposite ends of the linear chromosome.

The telomeres of this invention are useful in DNA linear plasmid constructs for use in producing transgenic plants by Agrobacterium tumefaciens transformation. Such plasmids comprise an origin of replication and covalently-closed terminal regions obtained from telomeres of this invention. Such plasmids should inherently advantageously limit the maximum amount of “border read-through”. In preferred aspects of the invention the use of such linear plasmids advantageously improves the efficiency and quality of Agrobacterium transformation. Preferred aspects of this invention provide such DNA linear plasmid constructs with telomeric ends and further comprising DNA segments selected from the group consisting of promoters, selectable markers, screenable markers and polypeptide-encoding sequence.

### BRIEF DESCRIPTION OF THE DRAWINGS

FIG. 1 illustrates restriction sites at the telomeres of the Agrobacterium tumefaciens linear chromosome and Southern blots of telomere fragments.

### DETAILED DESCRIPTION OF THE INVENTION

As used herein the term “plasmid” means an independently replicating, linear or circular piece of a DNA construct that can be transferred into an organism. The plasmids of this invention are preferably linear and capable of incorporating at least part of the DNA into the genome of the host organism.

As used herein, a nucleic acid molecule, be it a naturally occurring molecule or a fragment of a naturally occurring molecule or a synthetic molecule, may be “isolated”, if the molecule is separated from substantially all other molecules normally associated with it in its native state. More preferably an isolated molecule is substantially purified and is the predominant species present in a preparation. A substantially purified molecule may be greater than 60% free, preferably 75% free, more preferably 90% free, and most preferably 95% free from the other molecules (exclusive of solvent) present in the natural state. The term “isolated” is not intended to encompass molecules present in their natural or native state.

The telomeres and other nucleic acid molecules of this invention will preferably be “biologically active” with respect to facilitating DNA replication.

The agents of the present invention may also be recombinant. As used herein, the term recombinant describes (a) nucleic acid molecules that are constructed or modified outside of cells and that can replicate or function in a living cell, (b) molecules that result from the transcription, replication or translation of recombinant nucleic acid molecules, or (c) organisms that contain recombinant nucleic acid molecules or are modified using recombinant nucleic acid molecules.

The term “oligonucleotide” as used herein refers to short nucleic acid molecules useful, e.g., as hybridizing probes, nucleotide array elements, sequencing primers, or primers for DNA extension reactions, such as polymerase chain reaction. The size of the oligonucleotide molecules of the present invention will depend upon several factors, particularly on the ultimate function or use intended for a particular oligonucleotide. Oligonucleotides, i.e. deoxyribonucleotides or ribonucleotides, can comprise ligated natural nucleic acid molecules or synthesized nucleic acid molecules and will generally comprise between 15 to 1000 nucleotides or between about 20 and about 100 nucleotides. The sequence of the oligonucleotides will ideally be identical or complementary to the sequence of a fragment of similar length in an Agrobacterium nucleic acid molecule provided herein.

This invention provides oligonucleotides specific for nucleic acid molecules of the present invention. Such oligonucleotides find particular use as nucleic acid elements of solid arrays (e.g., synthesized or spotted), as hybridization probes, and as primers for amplification of telomeric regions of this invention. Oligonucleotides for use in polymerase chain reaction (PCR) as primers are preferably designed with the goal of amplifying nucleic acids from either the 3′ or the 5′ end of an Agrobacterium chromosome, e.g. about 500 to 800 bp of nucleic acids.

The term “primer” as used herein refers to a nucleic acid molecule, preferably an oligonucleotide whether derived from a naturally occurring molecule, such as one isolated from a restriction digest, or one produced synthetically, which is capable of acting as a point of initiation of synthesis when placed under conditions in which synthesis of a primer extension product which is complementary to a nucleic acid strand is induced, i.e., in the presence of nucleotides and an agent for polymerization such as DNA polymerase and at a suitable temperature and pH. The primer is preferably single stranded for maximum efficiency in amplification, but may alternatively be double stranded. If double stranded, the primer is first treated to separate its strands before being used to prepare extension products. Preferably, the primer is oligomeric DNA. The primer must be sufficiently long to prime the synthesis of extension products in the presence of the agent for polymerization. The exact lengths of the primers will depend on many factors, including temperature and source of primer. For example, depending on the complexity of the target sequence, the oligonucleotide primer typically contains at least 15, more preferably 18 nucleotides, which are identical or complementary to the template and optionally a tail of variable length which need not match the template. The length of the tail should not be so long that it interferes with the recognition of the template. Short primer molecules generally require cooler temperatures to form sufficiently stable hybrid complexes with the template.

The primers herein are selected to be “substantially” complementary to the different strands of each specific sequence to be amplified. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to be amplified to hybridize therewith and thereby form a template for synthesis of the extension product of the other primer. Computer generated search programs such as Primer3 (Steve Rozen, Helen J. Skaletsky (1996, 1997); code available at http://www.genome.wi.mit.edu/genome_software/other/primer3.html), STSPipeline (www-genome.wi.mit.edu/cgi-bin/www-STS_Pipeline), or GeneUp (Pesole et al., BioTechniques 25:112-123 (1998)), for example, can be used to identify potential PCR primers. Exemplary primers include primers that are 18 to 50 bases long, where at least between 18 to 25 bases are identical or complementary to a segment of corresponding length in the template sequence.

Nucleic acid molecules or fragments thereof are capable of hybridizing to other nucleic acid molecules under certain circumstances. As used herein, two nucleic acid molecules are said to be capable of hybridizing to one another if the two molecules are capable of forming an anti-parallel, double-stranded nucleic acid structure. A nucleic acid molecule is said to be the “complement” of another nucleic acid molecule if they exhibit “complete complementarity” i.e. each nucleotide in one sequence is complementary to its base pairing partner nucleotide in another sequence. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., Molecular Cloning, A Laboratory Manual, 2nd Ed., Cold Spring Harbor Press, Cold Spring Harbor, N.Y. (1989), and by Haymes et al., Nucleic Acid Hybridization, A Practical Approach, IRL Press, Washington, D.C. (1985), the entirety of both of which are herein incorporated by reference. Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed.

Appropriate stringency conditions which promote DNA hybridization, for example, incubation in 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

Preferred embodiments of the nucleic acid of this invention will hybridize to one or more of the nucleic acid molecules of this invention or complements thereof under low stringency conditions, for example at about 2.0×SSC and about 50° C. In a particularly preferred embodiment, a nucleic acid of the present invention will include those nucleic acid molecules that hybridize to one or more of the nucleic acid molecules of this invention or complements thereof under moderate stringency conditions. In an especially preferred embodiment, a nucleic acid of the present invention will include those nucleic acid molecules that hybridize to one or more of the nucleic acid molecules of this invention or complements thereof under high stringency conditions.

As used herein “sequence identity” refers to the extent to which two optimally aligned polynucleotide or polypeptide sequences are invariant throughout a window of alignment of components, e.g. nucleotides or amino acids. An “identity fraction” for aligned segments of a test sequence and a reference sequence is the number of identical components which are shared by the two aligned sequences divided by the total number of components in reference sequence segment, i.e. the entire reference sequence or a smaller defined part of the reference sequence. “Percent identity” is the identity fraction times 100.

Useful methods for determining sequence identity are disclosed in “Guide to Huge Computers”, Martin J. Bishop, ed., Academic Press, San Diego, 1994, and Carillo, H., and Lipton, D., SIAM J Applied Math (1988) 48:1073, each of which is incorporated herein by reference. More particularly, preferred computer programs for determining sequence identity include the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. Mol. Biol. 215:403-410 (1990), incorporated herein by reference; version 2.0 or higher of BLAST programs allows the introduction of gaps (deletions and insertions) into alignments; for polypeptide sequence BLASTX can be used to determine sequence identity; and, for polynucleotide sequence BLASTN can be used to determine sequence identity.

For purposes of this invention “percent identity” shall be determined using BLASTX version 2.0.08 for nucleotide translations of polypeptide sequences and BLASTN version 2.0.08 for polynucleotide sequences.

DNA Constructs

The present invention also encompasses the use of telomeres of the present invention in recombinant constructs. Using methods known to those of ordinary skill in the art, telomeres of this invention can be inserted into constructs also comprising an origin of replication and polypeptide-encoding sequence operably linked to a promoter. Such constructs can be introduced into a host cell of choice for expression of the encoded polypeptide. Potential host cells include both prokaryotic and eukaryotic cells. A host cell may be unicellular or found in a multicellular differentiated or undifferentiated organism depending upon the intended use. It is understood that useful exogenous genetic material may be introduced into any cell or organism such as a bacterial cell, fungal cell, fungus, plant cell, plant, mammalian cell, mammal, fish cell, fish, bird cell, bird or bacterial cell. Plant cells are a preferred target host organism.

Depending upon the host, the regulatory regions for expression of transgenic DNA will vary and may include regions from viral, plasmid or chromosomal genes, or the like. For expression in prokaryotic or eukaryotic microorganisms, particularly unicellular hosts, a wide variety of constitutive or regulatable promoters may be employed. Among transcriptional initiation regions which have been described are those obtained from bacterial and yeast hosts, such as E. coli, B. subtilis, and Sacchromyces cerevisiae, including genes such as beta-galactosidase, T7 polymerase and tryptophan E.

Furthermore, for use in transformation of A. tumefaciens, constructs may include those in which a protein encoding sequence is positioned with respect to a promoter sequence such that production of antisense mRNA complementary to native mRNA molecules is provided. In this manner, expression of a native gene may be decreased. Such methods may find use for modification of particular functions of the targeted host, and/or for discovering the function of a naturally expressed protein.

The present invention also encompasses the use of nucleic acid constructs of the present invention in constructs used for mutation of genes within A. tumefaciens using homologous recombination, e.g., as disclosed by Lloyd et al. in Chapter 119 of “Escherichia coli and Salmonella—cellular and molecular biology”, Second Edition, © 1996 by ASM Press, Washington D.C. Such constructs, for example, may contain two encoding segments of a protein encoding sequence harboring a heterologous portion of DNA (such as an antibiotic resistance marker) between the two encoding segments. Such constructs may also contain, for example, other deletions, insertions, or base changes, or combinations thereof, relative to the A. tumefaciens-derived telomeric sequence. Introduction of these constructs into A. tumefaciens can be used to generate mutations in the DNA of A. tumefaciens. Such mutations are useful, for example, in functional analysis of the mutated genes.

As used herein, a promoter region is a region of a nucleic acid molecule that is capable, when located cis to a nucleic acid sequence that encodes for a protein or polypeptide to function in a way that directs transcription of one or more mRNA molecules that encodes for the protein or polypeptide. Promoters may be located directly 5′ of the protein encoding sequence, for example where a promoter regulates transcription of a single gene. Alternatively, such as when a promoter regulates transcription of a group of genes in an operon, the promoter may be located some distance upstream from a particular encoding region. Promoters will generally be recognized by their presence 5′, or upstream, of the start site for a protein coding region and/or by the presence of the −10 and −35 consensus core promoter elements found in bacterial promoters. In addition, promoters may contain additional non-core sequences which can affect promoter strength. Such additional regulatory sequences may be located upstream of, downstream of, or between core promoter elements. Examples of additional regulatory elements include UP elements (−40 upstream region) and DSR elements (region immediately downstream of the transcription start site).

The deduced structure of the linear chromosome suggests that it contains one origin of replication (a repABC-type replication system), and replication termini at each telomere. Due to its linear nature, the mode of replication of the linear plasmid will differ significantly from the mode of replication of the circular chromosome, with a specialized mechanism for replicating the chromosome termini. The telomeres at the chromosome termini provide the special structures needed for complete replication, and eventually separation, of the daughter chromosomes.

The recombinant vector of this invention can be a single linear plasmid or a system of additional plasmids which together contain the total DNA to be introduced into the genome of the host. Methods which can be used to introduce recombinant vectors into Agrobacterium species include triparental mating (Ditta et al. (1985) Plasmid 13:149-153; Ditta et al. (1980) Proc. Natl. Acad. Sci. USA 77:7347-7351), electroporation (White et al. (1995) Meth. in Mol. Biol. 47:135-141) and P1 transduction (Avery L. and Kaiser D. (1983) Mol. Gen. Genet. 191:99-109).

The constructs of the present invention preferably contain one or more selectable markers which permit easy selection of transformed cells. A selectable marker is a gene whose product provides, for example, biocide or viral resistance, resistance to heavy metals, prototrophy to auxotrophs, and the like. Various selectable markers may be used depending upon the host species to be transformed, and different conditions for selection may be used for different hosts.

A construct of this invention may comprise polypeptide encoding sequence operably linked to a suitable promoter sequence and optionally to a suitable leader sequence. A leader sequence may be a nontranslated region of an mRNA which is important for translation by a host cell. A leader sequence is operably linked to the 5′ terminus of the nucleic acid sequence encoding the polypeptide. The leader sequence may be native to the nucleic acid sequence encoding the polypeptide or may be obtained from foreign sources. A polyadenylation sequence may also be operably linked to the 3′ terminus of the nucleic acid sequence of the present invention, particularly for use in eukaryotic host cells.

To avoid the necessity of disrupting the cell to obtain the polypeptide, and to minimize the amount of possible degradation of the expressed polypeptide within the cell, it may be preferred that expression of the polypeptide gives rise to a product secreted outside the cell, especially in the case of expression in bacterial host cells of bacterium or bacteria. To this end, the polypeptide of the present invention may be linked to a signal peptide linked to the amino terminus of the polypeptide. A signal peptide is an amino acid sequence which permits the secretion of the polypeptide from the host into the culture medium.

A nucleic acid molecule of the present invention which encodes a polypeptide may also be linked to a propeptide coding region. A propeptide is an amino acid sequence found at the amino terminus of apoprotein or proenzyme. Cleavage of the propeptide from the proprotein yields a mature biochemically active protein. The resulting polypeptide is known as a propolypeptide or proenzyme (or a zymogen in some cases). Propolypeptides are generally inactive and can be converted to mature active polypeptides by catalytic or autocatalytic cleavage of the propeptide from the propolypeptide or proenzyme. The propeptide coding region may be native to the polypeptide or may be obtained from foreign sources.

A nucleic acid molecule of the present invention which encodes a polypeptide may also be linked to a transit peptide coding region. A transit peptide is an amino acid sequence found at the amino terminus of an active protein which provides for transport of the protein into a plastid organelle, such as a plant chloroplast. The transit peptide coding region may be native to the type of cell to be transformed, or may be obtained from foreign sources.

Plant Constructs and Plant Transformants

Of particular interest is the use of DNA constructs of this invention for plant transformation or transfection. Exogenous genetic material may be transferred into a plant cell and the plant cell regenerated into a whole, fertile or sterile plant. Exogenous genetic material is any genetic material, whether naturally occurring or otherwise, from any source that is capable of being inserted into any organism. Such genetic material may be transferred into either monocotyledons and dicotyledons including but not limited to the plants, alfalfa, Arabidopsis thaliana, barley, broccoli, cabbage, citrus, cotton, garlic, oat, oilseed rape (canola), onion, flax, maize, ornamental plants, pea, peanut, pepper, potato, rice, rye, sorghum, soybean, strawberry, sugarcane, sugarbeet, tomato, wheat, poplar, pine, fir, eucalyptus, apple, potato, lettuce, lentils, grape, banana, tea, turf grasses, sunflower, oil palm, etc.

Many different methods for generating transgenic plants using A. tumefaciens have been described. In general, these methods rely on a “disarmed” A. tumefaciens strain that is incapable of inducing tumors, and a binary plasmid transfer system. The disarmed strain has the oncogenic genes of the T-DNA deleted. A binary plasmid transfer system consists of one plasmid with short, e.g. 23-25 base pair, T-DNA left and right border sequences, between which a gene for a selectable marker (e.g., an herbicide resistance gene) and other desired genetic elements are cloned and a second plasmid which encodes the A. tumefaciens genes necessary for effecting the transfer of the DNA between the border sequences in the first plasmid. When plant tissue is exposed to Agrobacterium carrying the two plasmids, the DNA between the left and right border repeats is transferred into the plant cells, transformed cells are identified using the selectable marker, and whole plants are regenerated from the transformed tissue. Plant tissue types that have been reported to be transformed using variations of this method include: cultured protoplasts (Komari, T., 1989, Plant Science, 60:223-229), leaf disks (Lloyd, A. M. et al., 1986, Science 234:464-466), shoot apices (Gould, J., et al., 1991, Plant Physiology, 95:426-434), root segments (Valvekens, D. et al., 1988, PNAS, 85:5536-5540), tuber disks (Jin, S. et al., 1987, Journal of Bacteriology, 169: 4417-4425), and embryos (Gordon-Kamm W., et al., 1990, Plant Cell, 2:603-618).

In the case of Arabidopsis thaliana it is possible to perform in planta germline transformation (Katavic B., et al., 1994, Molecular and General Genetics, 245:363-370; (Clough, S. and Bent, A., 1998, Plant Journal, 16:735-743). In the simplest of these methods, flowering Arabidopsis plants are dipped into a culture of Agrobacterium such as that described in the previous paragraph. Among the seeds produced from these plants, 1% or more have integration of T-DNA into the genome.

Monocot plants have generally been more difficult to transform with Agrobacterium than dicot plants. However, “supervirulent” strains of Agrobacterium with increased expression of the virB and virG genes have been reported to transform monocot plants with increased efficiency (Komari T. et al., 1986, Journal of Bacteriology, 166:88-94; Jin S., et al., 1987, Journal of Bacteriology, 169:417-425).

Most T-DNA insertion events are due to illegitimate recombination events and are targeted to random sites in the genome. However, given sufficient homology between the transferred DNA and genomic sequence, it has been reported that integration of T-DNA by homologous recombination may be obtained at a very low frequency. Even with long stretches of DNA homology, the frequency of integration by homologous recombination relative to integration by illegitimate recombination is roughly 1:1000 (Miao, Z. and Lam, E., 1995, Plant Journal, 7:359-365; Kempin S. A. et al., 1997, 389:802-803).

Exogenous genetic material may be transferred into a plant cell by the use of a DNA vector or construct designed for such a purpose. Vectors have been engineered for transformation of large DNA inserts into plant genomes. Binary bacterial artificial chromosomes have been designed to replicate in both E. coli and A. tumefaciens and have all of the features required for transferring large inserts of DNA into plant chromosomes. BAC vectors, e.g., a pBACwich, have been developed to achieve site-directed integration of DNA into a genome.

A construct or vector may also include a plant promoter to express the protein or protein fragment of choice. A number of promoters which are active in plant cells have been described in the literature. These include the nopaline synthase (NOS) promoter, the octopine synthase (OCS) promoter, a caulimovirus promoter such as the CaMV 19S promoter and the CaMV 35S promoter, the figwort mosaic virus 35S promoter, the light-inducible promoter from the small subunit of ribulose-1,5-bis-phosphate carboxylase (ssRUBISCO), the Adh promoter, the sucrose synthase promoter, the R gene complex promoter, and the chlorophyll a/b binding protein gene promoter. For the purpose of expression in source tissues of the plant, such as the leaf, seed, root or stem, it is preferred that the promoters utilized in the present invention have relatively high expression in these specific tissues. For this purpose, one may choose from a number of promoters for genes with tissue- or cell-specific or -enhanced expression. Examples of such promoters reported in the literature include the chloroplast glutamine synthetase GS2 promoter from pea, the chloroplast fructose-1,6-biphosphatase (FBPase) promoter from wheat, the nuclear photosynthetic ST-LS1 promoter from potato, the phenylalanine ammonia-lyase (PAL) promoter and the chalcone synthase (CHS) promoter from Arabidopsis thaliana. Also reported to be active in photosynthetically active tissues are the ribulose-1,5-bisphosphate carboxylase (RbcS) promoter from eastern larch (Larix laricina), the promoter for the cab gene, cab6, from pine, the promoter for the Cab-1 gene from wheat, the promoter for the CAB-1 gene from spinach, the promoter for the cab1R gene from rice, the pyruvate, orthophosphate dikinase (PPDK) promoter from Zea mays, the promoter for the tobacco Lhcb gene, the Arabidopsis thaliana SUC2 sucrose-H+symporter promoter, and the promoter for the thylacoid membrane proteins from spinach (psaD, psaF, psaE, PC, FNR, atpC, atpD, cab, rbcS). Other promoters for the chlorophyl a/b-binding proteins may also be utilized in the present invention, such as the promoters for LhcB gene and PsbP gene from white mustard (Sinapis alba). Additional promoters that may be utilized are described, for example, in U.S. Pat. Nos. 5,378,619; 5,391,725; 5,428,147; 5,447,858; 5,608,144; 5,608,144; 5,614,399; 5,633,441; 5,633,435.

Constructs or vectors may also include, with the coding region of interest, a nucleic acid sequence that acts, in whole or in part, to terminate transcription of that region. For example, such sequences have been isolated including the Tr7 3′ sequence and the nos 3′ sequence or the like. It is understood that one or more sequences of the present invention that act to terminate transcription may be used.

A vector or construct may also include other regulatory elements or selectable markers. Selectable markers may also be used to select for plants or plant cells that contain the exogenous genetic material. Examples of such include, but are not limited to, a neo gene which codes for kanamycin resistance and can be selected for using kanamycin, G418, etc.; a bar gene which codes for bialaphos resistance; a mutant EPSP synthase gene which encodes glyphosate resistance; a nitrilase gene which confers resistance to bromoxynil, a mutant acetolactate synthase gene (ALS) which confers imidazolinone or sulphonylurea resistance; and a methotrexate resistant DHFR gene.

A vector or construct may also include a screenable marker to monitor expression. Exemplary screenable markers include a b-glucuronidase or uidA gene (GUS), an R-locus gene, which encodes a product that regulates the production of anthocyanin pigments (red color) in plant tissues; a b-lactamase gene, a gene which encodes an enzyme for which various chromogenic substrates are known (e.g., PADAC, a chromogenic cephalosporin); a luciferase gene, a xylE gene which encodes a catechol dioxygenase that can convert chromogenic catechols; an a-amylase gene, a tyrosinase gene which encodes an enzyme capable of oxidizing tyrosine to DOPA and dopaquinone which in turn condenses to melanin; an a-galactosidase, which will turn a chromogenic a-galactose substrate. Included within the terms “selectable or screenable marker genes” are also genes which encode a secretable marker whose secretion can be detected as a means of identifying or selecting for transformed cells. Examples include markers which encode a secretable antigen that can be identified by antibody interaction, or even secretable enzymes which can be detected catalytically. Secretable proteins fall into a number of classes, including small, diffusible proteins detectable, e.g., by ELISA, small active enzymes detectable in extracellular solution (e.g., a-amylase, b-lactamase, phosphinothricin transferase), or proteins which are inserted or trapped in the cell wall (such as proteins which include a leader sequence such as that found in the expression unit of extension or tobacco PR-S). Other possible selectable and/or screenable marker genes will be apparent to those of skill in the art.

### EXAMPLE 1

This example illustrates obtaining and characterizing isolated telomeres from the ends of the linear chromosome of A. tumefaciens. The genomic DNA sequence of the linear chromosome are derived from a double stranded library. The two basic methods for the DNA sequencing are the chain termination method of Sanger et al., Proc. Natl. Acad. Sci. (U.S.A.) 74:5463-5467 (1977) and the chemical degradation method of Maxam and Gilbert, Proc. Natl. Acad. Sci. (U.S.A.) 74:560-564 (1977) using automated fluorescence-based sequencing as reported by Craxton, Method, 2:20-26 (1991); Ju et al., Proc. Natl. Acad. Sci. (U.S.A.) 92:4347-4351 (1995); and Tabor and Richardson, Proc. Natl. Acad. Sci. (U.S.A.) 92:6339-6343 (1995) and high speed capillary gel electrophoresis, e.g., as disclosed by Swerdlow and Gesteland, Nucleic Acids Res. 18:1415-1419 (1990); Smith, Nature 349:812-813 (1991); Luckey et al., Methods Enzymol. 218:154-172 (1993); Lu et al., J. Chromatog. A. 680:497-501 (1994); Carson et al., Anal. Chem. 65:3219-3226 (1993); Huang et al., Anal. Chem. 64:2149-2154 (1992); Kheterpal et al., Electrophoresis 17:1852-1859 (1996); Quesada and Zhang, Electrophoresis 17:1841-1851 (1996); Baba, Yakugaku Zasshi 117:265-281 (1997). For instance, genomic nucleotide sequence traces are generated using a 377 DNA Sequencer (Perkin-Elmer Corp., Applied Biosystems Div., Foster City, Calif.) allowing for rapid electrophoresis and data collection. With these types of automated systems, fluorescent dye-labeled sequence reaction products are detected and chromatograms are subsequently viewed, stored in a computer and analyzed using corresponding apparatus-related software programs. These methods are known to those of skill in the art and have been described and reviewed (Birren et al., Genome Analysis: Analyzing DNA, 1, Cold Spring Harbor, N.Y. (1998)).

Quality genomic sequence traces are assembled generally as follows:

(a) all traces are “vector-trimmed’ i.e., 5′ and 3′ vector and linker sequences are removed;

(b) a PHRAP assembly is run using default assembly parameters;

(c) contigs and singletons files and their corresponding quality files are united to create “islands” of contiguous sequence (contigs) from which genes are identified by sequence query against known gene databases.

After genes are identified there is remaining DNA comprising the telomeric regions. The telomeric region at the left end of the linear chromosome comprises DNA in the sequence of SEQ ID NO: 1. The other telomeric region at the right end of the linear chromosome comprises DNA in the sequence of SEQ ID NO: 2.

The telomeric region can be amplified by PCR using probes designed from SEQ ID NOs: 1 and 2. The telomeric regions can be sequenced. Alternatively, smaller telomeric regions can be isolated by cutting away DNA running toward the middle of the chromosome using a restriction enzyme which is selected to match a restriction site in SEQ ID NO: 1 or 2. Oligonucleotide probes having the sequence of SEQ ID NOs: 3 and 4 are used in a hybridization gel for Southern blots to identify progressively smaller restriction fragments of telomeric region comprising SEQ ID NOs: 1 and 2. In particular, restriction enzyme Kpn I can be used to cut a telomeric region of the left end of the linear chromosome which hybridizes to a probe of SEQ ID NO: 3; and restriction enzyme Eco RI can be used to cut a telomeric region of the right end of the linear chromosome which hybridizes to a probe of SEQ ID NO: 4.

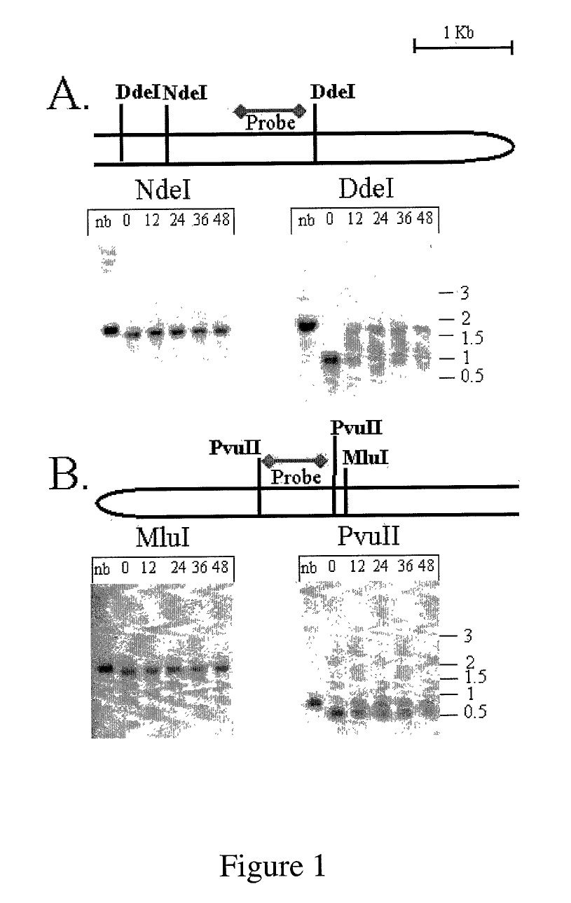

More particularly with reference to FIG. 1 there is shown restriction sites for the telomeres of the linear chromosome and Southern blots of the restriction fragments. Southern blot hybridization was performed on DNA fragments containing the Right (A) and Left (B) telomeres of the linear chromosome. Probes recognized DNA very near each telomere, and these were used to detect DNA fragments containing either intact telomeres (NdeI and MluI), or fragments lacking the telomere ends (DdeI and PvuII). A portion of the DNA was boiled for 10 minutes and allowed to cool slowly for various periods of time. DNA fragments were separated by agarose gel electrophoresis, then transferred to nylon membranes prior to hybridization. The mobility of fragments containing the telomeres was essentially identical regardless of whether the DNA samples had been denatered. These data indicate that the two strands of DNA containing the telomeres are covalently closed. Fragments lacking the telomere ends migrated faster following boiling, indicating denaturation creating two single-stranded DNA molecules. Slow cooling allowed renaturation of a portion of the denatured molecules. nb, not boiled; 0, 12, 24, 36 and 48 indicate the number of minutes that the DNA samples were allowed to cool before they were frozen in a dry ice-ethanol bath. After 48 minutes of cooling, the temparature of the DNA samples was approximately 50° C. Numbers on the right of each figure indicated the size, in kilobases, of a double-stranded DNA molecular weight standard. The Southern blots of denatured DNA fragments containing either telomere show a single molecule rather than two single-stranded molecules; the single fragments indicate that the two strands near the telomere are joined by a hairpin loop.

The complete sequence of the telomeres can be deduced by using the DNA sequence present in SEQ ID NO:1 or 2 to prime PCR products that extend around the covalently-closed telomeres of the linear chromosome. The resulting DNA product may either be sequenced directly, or cloned and sequenced as described above. Alternatively, the sequence may be read directly from the chromosomal DNA of Agrobacterium, or from partially purified fragments thereof, as practiced by Fidelity Systems, Inc. (Gaithersburg, Md.). The proximal regions of both telomeres are similar in overall architecture, but are very different in sequence. The sequence near both telomeres contains several IS elements, with intervening DNA of additional repeated and unique sequence. The region is rich in potential secondary structure and contains numerous short sequence repeats.

### EXAMPLE 2

This example serves to illustrate the design of linear plasmid constructs of this invention. A linear plasmid is constructed comprising in order, a left telomere region of this invention, an origin of replication from A. tumefaciens, a promoter region, a polypeptide encoding region, a polyadenylation region, a selectable marker region, a screenable marker region and a right telomeric region of this invention. A construct may also be viable if it contains identical telomeres at both ends. Such a construct is used by transforming Agrobacterium and employing methods well known in the art to make transgenic plants such as of cotton, corn and soybean.

All publications and patent applications are herein incorporated by reference in their entirely to the same extent as if each individual publication or patent application was specifically and individually indicated to be incorporated by reference.

Although the invention has been described in some detail by way of illustration and example for purposes of clarity of understanding, it will be obvious that certain changes and modifications may be practiced within the scope of the appended claims.

What is claimed is:

1. An isolated telomere from the linear chromosome of an Agrobacterium tumefaciens
wherein said telomere is obtainable from a restriction enzyme fragment at the end of said chromosome;
wherein said fragment comprises less than 4,000 nucleotide bases and comprises a segment of consecutive nucleotide bases having at least 90% identity to SEQ ID NO: 1 or SEQ ID NO: 2;
and wherein said telomere is obtained by removing at least said segment from said fragment.

2. An isolated telomere according to claim 1 comprising a covalently-closed end.

3. An isolated telomere according to claim 1 wherein said consecutive nucleotide bases have at least 95% identity to SEQ ID NO: 1 or SEQ ID NO: 2.

4. An isolated telomere according to claim 1 wherein the restriction enzyme producing the fragment comprising SEQ ID NO: 1 is Kpn I.

5. An isolated telomere according to claim 1 wherein the restriction enzyme producing the fragment comprising SEQ ID NO: 2 is Eco RI.

6. A pair of isolated and distinct telomeres obtained from opposite ends of said linear chromosome wherein each of said telomeres has a nucleic acid sequence of a telomere of claim 1.

7. An isolated telomere from the linear chromosome of an Agrobacterium tumefaciens having a covalently-closed end,
wherein said telomere is obtainable from a restriction enzyme fragment at the end of said chromosome;
wherein said fragment comprises less than 4,000 nucleotide bases and comprises a segment of consecutive nucleotide bases having at least 90% identity to SEQ ID NO: 1 or SEQ ID NO: 2;
and wherein said telomere is obtained by removing at least said segment from said fragment.

8. A linear DNA construct for use in producing transgenic plants by Agrobacterium tumefaciens transformation, said construct comprising at least an origin of replication and terminal regions obtained from telomeres of claim 1.

9. A linear DNA construct according to claim 8 further comprising at least one DNA segment selected from the group consisting of promoters and selectable markers.

10. A linear DNA construct according to claim 9 having covalently-closed ends.
